(19) United States
(12) Patent Application Publication
(10) Pub. No.: US 2026/0120473 A1
(43) Pub. Date: Apr. 30, 2026

(54) SURGICAL ANALYSIS AND SCORING SYSTEM

(71) Applicant: Kaliber Labs Inc., San Francisco, CA (US)

(72) Inventors: Chandra JONELAGADDA, San Francisco, CA (US); Aneesh JONELAGADDA, San Francisco, CA (US); Salvatore PENACHIO, San Francisco, CA (US)

(21) Appl. No.: 19/473,479

(22) PCT Filed: Sep. 25, 2024
(86) PCT No.: PCT/US2024/048435

§ 371 Date: Oct. 7, 2025

Related U.S. Application Data
(60) Provisional application No. 63/585,950, filed on Sep. 27, 2023.

(51) Int. Cl. G06V 20/52; A61B 34/00; G06V 10/82; G06V 20/40; G06V 20/70

(52) U.S. Cl. CPC G06V 20/52; A61B 34/00; G06V 10/82; G06V 20/40; G06V 20/70; G06V 2201/03

(57) ABSTRACT

Apparatuses, systems, and methods to assess a surgeon's performance based on a surgical video recording or live-stream of the surgeon performing an actual procedure may be analyzed using a variety of trained neural networks (e.g., machine-learning based techniques). In some examples, the assessment can include one or more graphical representations (graphs) that illustrate and summarize the surgeon's behavior and actions during a surgical procedure. The graphs can enable other doctors to assess and coach a surgeon's performance.

### CLAIM OF PRIORITY

This patent application claims priority to U.S. provisional patent application No. 63/585,950, titled “SURGICAL ANALYSIS AND SCORING SYSTEM,” filed Sep. 27, 2023, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present embodiments relate generally to surgery and more specifically to providing a method and/or apparatus for assessment of surgical proficiency.

### BACKGROUND

Assessment of surgical proficiency is conventionally a manual process. Surgeons in academic institutions provide feedback to the surgeon fellows by observing the surgeries in real-time or by reviewing the surgical videos. There is considerable subjectivity in these assessments. Surgeon fellows are unable to properly ascertain any improvements they might have made in the time between assessments. Furthermore, once the surgeons graduate medical school, it becomes virtually impossible to assess their performance in the field. The surgeons' skill may become stale or out of date.

### SUMMARY OF THE DISCLOSURE

Described herein are apparatuses, systems, and methods to assess a surgeon's performance. The assessment can be based on a surgical video recording or live-stream of the surgeon performing an actual procedure. The recorded video or live-stream may be analyzed using a variety of trained neural networks (e.g., machine-learning based techniques). In some examples, the assessment can include one or more graphical representations (graphs) that illustrate and summarize the surgeon's behavior and actions during a surgical procedure. The graphs can enable other doctors to assess and coach a surgeon's performance.

Any of the methods described herein may provide a surgical assessment. The method may include receiving a video of a surgical procedure, wherein the video includes a plurality of frames, each frame having a unique timestamp. The method may further include generating intermediate assessment data from the video of the surgical procedure based on an execution of one or more trained neural networks configured to recognize one or more clinical aspects within the video, wherein the intermediate assessment data includes one or more text labels identifying the clinical aspect, and interactively displaying a graphical representation of surgical information included in the video, based on the intermediate assessment data.

In general, the graphical representation may include a graph, such as a line and/or a bar graph. The graphs may distill large amounts of information and data into a form more easily reviewed and scrutinized by subject matter experts.

In any of the methods described herein, the intermediate assessment data can include a text label indicating a recognized surgical procedure and a timestamp of the video that includes the recognized surgical procedure. Since the intermediate assessment data may be provided by an execution of a trained neural network, the trained neural network can be trained to provide the text labels as sell as a timestamp of the surgical video that is associated with the text label.

In any of the methods described herein, interactively displaying can include receiving a user input to display more detail with respect to at least one region of the graphical representation, and expanding a displayed region of the graphical representation in response to the user input.

In any of the methods described herein, the intermediate assessment data can include a text label indicating a recognized implant within the video and a timestamp of the video that includes the recognized implant.

In some examples, the methods described herein the intermediate assessment data can include a text label that indicates a recognized surgical tool within the surgical video and a timestamp of the video that includes the recognized surgical tool. In some cases, the intermediate assessment data further includes a mask identifying the surgical tool in the video.

In any of the methods described herein, the intermediate assessment data can include a text label identifying a recognized surgical location and a timestamp of the video that includes the recognized surgical location. In some embodiments, the intermediate assessment data can further include a mask identifying an area of pathology in the video.

In any of the methods described herein the intermediate assessment data can include a text label indicating a recognized anatomy and a timestamp of the video that includes the recognized anatomy. In some cases, the intermediate assessment data further includes a mask identifying the recognized anatomy in the surgical video.

In any of the methods described herein, the surgical information indicates a time of appearance of an identified surgical tool within the video. Furthermore, in any of the methods described herein, the surgical information indicates a time of appearance of an identified anatomy. In some examples, the identified anatomy may include two or more identified anatomies. In some other examples, the identified anatomy is expressed as a Boolean value.

In any of the methods described herein, the surgical information may indicate or include a monocular depth estimation. In some cases, the intermediate assessment data can include image quality evaluations. In general image quality evaluations may include descriptors of blurry output, bloody video scenes, and/or excessive debris. For example, the image quality evaluations can include evaluations of blur, color balance, loose body detection, or a combination thereof.

Any of the methods described herein can include detecting the presence of a surgical tool within a region not intended for its use. In any of the methods described herein may include detecting bleeding within the video. The presence of blood may indicate a suboptimal surgical procedure.

Any of the methods described herein may include detecting a failure to progress with respect to a surgical procedure. A failure to progress characteristic may indicate a stalled procedure or reluctance to proceed on the part of the surgeon.

Any of the methods described herein may include a time to pathology determination that indicates a time between detection of a pathology and detection of a specific tool within the video. In some examples, the methods described herein may include a tool change efficiency analysis based on labels of tools within the intermediate assessment data.

In any system described herein may include one or more processors, a memory configured to store instructions that, when executed by the one or more processors, cause the system to: receive a video of a surgical procedure, wherein the video includes a plurality of frames, each frame having a unique timestamp, receive a video of a surgical procedure, wherein the video includes a plurality of frames, each frame having a unique timestamp, and interactively display a graphical representation of surgical information included in the video, based on the intermediate assessment data.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart depicting an example method for analyzing and reviewing a surgical video.

FIG. 2 shows a block diagram of primary analysis modules that may be associated with the primary analysis of a surgical video.

FIGS. 3A and 3B show example structures corresponding anatomical structure heatmaps.

FIG. 4 shows a block diagram of a secondary analysis.

FIG. 5 shows an example graphical representation based on a multi-dimensional vector described herein with respect to a shoulder procedure.

FIGS. 6A and 6B show example graphical representations of surgical tool use with respect to time.

FIG. 7 is an example of a graphical representation for a surgical procedure in the knee (meniscal repair).

FIG. 8 is an example graphical representation of a surgical procedure for anterior cruciate ligament reconstruction.

FIG. 9 shows a block diagram of a device that may perform some or all of the operations described herein.

### DETAILED DESCRIPTION

Analysis and review of surgical procedures may be performed by an automated agent that receives, as input, a video recording, or a video stream of a surgical procedure. In some examples, the automated agent, which may be realized by a system, apparatus, and/or processor executing a non-transitory computer-readable medium, can return scores and insights associated with a detected surgical procedure. The surgeon performing the surgical procedure can review the scores and insights and use them to improve or refine his or her surgical technique.

In some embodiments, the scores and insights may be provided through the execution of one or more trained neural networks. The neural networks can review multiple aspects of a surgical procedure either independently or in combination with other neural networks and provide feedback to the surgeon. In this manner, a surgeon can receive feedback on surgical techniques and procedures based on performing actual procedures. Conventionally, such feedback was limited to teaching or monitored procedures. Through one or more of the embodiments described herein, the feedback may be proved to any practitioner in any capacity and not limited to teaching situations or any monitored procedures.

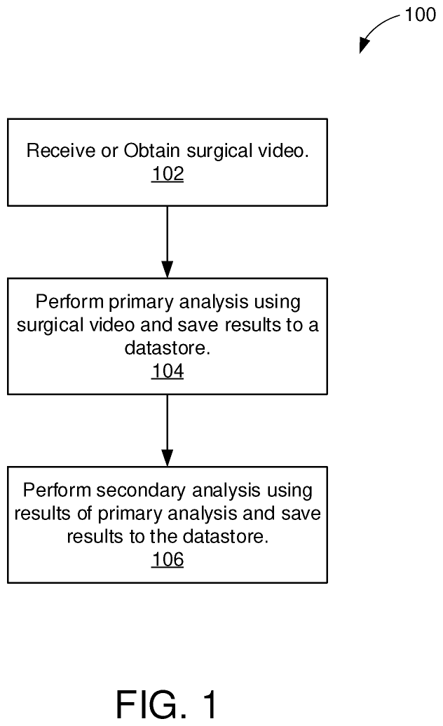

FIG. 1 is a flowchart depicting an example method 100 for analyzing and reviewing a surgical video. In general a surgical video can be an feasible video of a surgical procedure. Some examples may perform the operations described herein with additional operations, fewer operations, operations in a different order, operations in parallel, and some operations differently. The method 100 may be used to review surgical procedures performed by a surgeon or other clinician. The method 100 can create feedback that enables the surgeon to identify areas of improvement and also measure improvement over time.

The method 100 may begin in block 102 as a surgical video is received or obtained. For example, the surgeon may provide a file including a video recording of a surgical procedure that was performed by the surgeon. In another example, the surgeon may provide a real-time video stream of surgical procedure. In some cases, the associated surgical procedure may have occurred or be occurring in a separate location. For example, some or all of the procedures described by the method 100 may be performed in a cloud-based server or similar environment. Thus, any hardware or other computing device used to perform the method 100 may be physically separated from the surgical venue. In some embodiments, the received or obtained video may be stored in a memory, datastore, database, or other feasible data storage device.

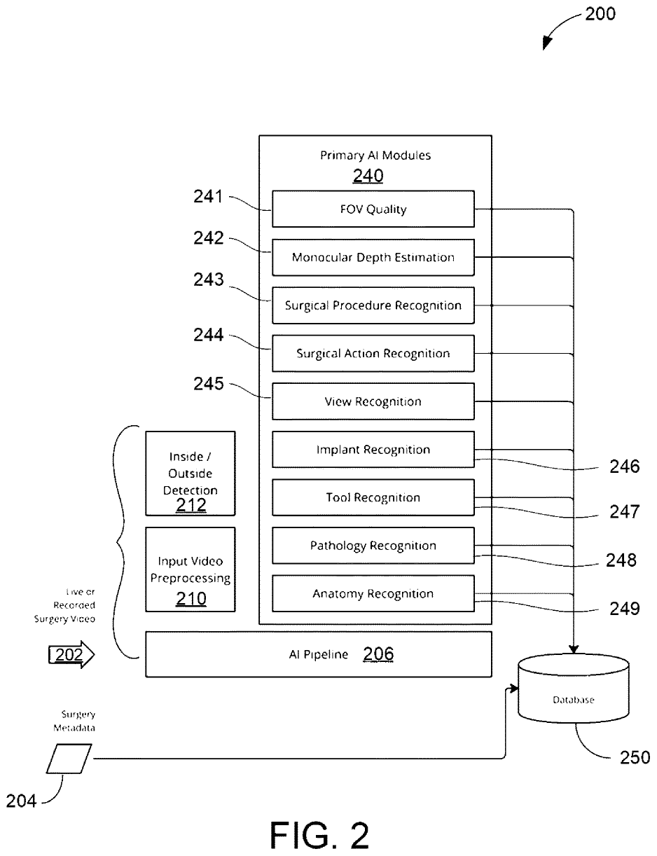

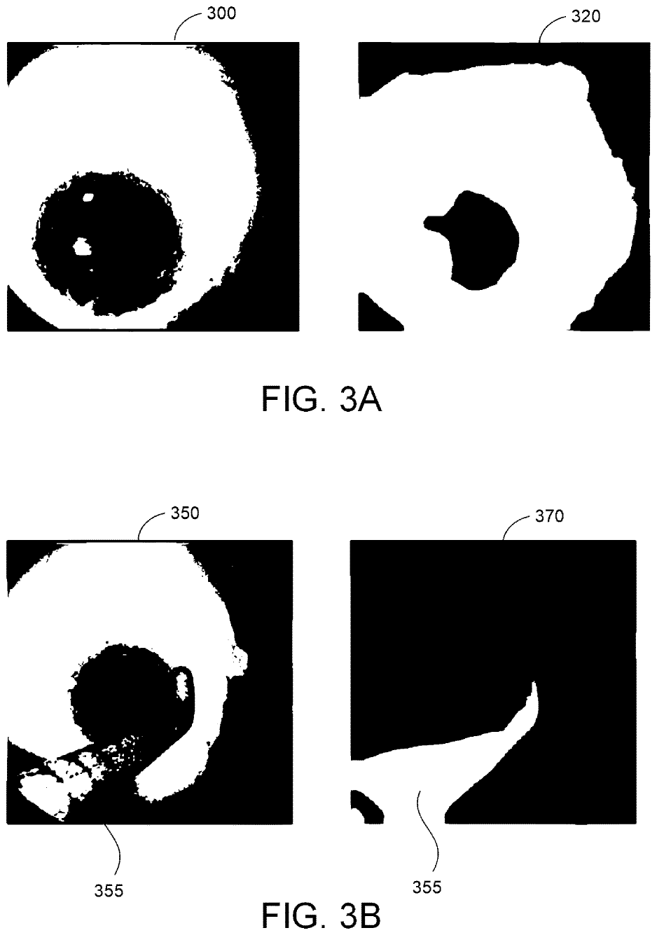

Next, in block 104, a primary analysis of the surgical video (or video stream) is performed. In some examples, the primary analysis may include analysis by one or more processors executing one or more trained neural networks. The trained neural network may provide a first level or for first-pass analysis of the information that may be included in the received surgical video or video stream. The results of the primary analysis may be stored in the memory, datastore, database, or other feasible storage device similar to as described with respect to block 102. Primary analysis is described in more detail below in conjunction with FIGS. 2-3.

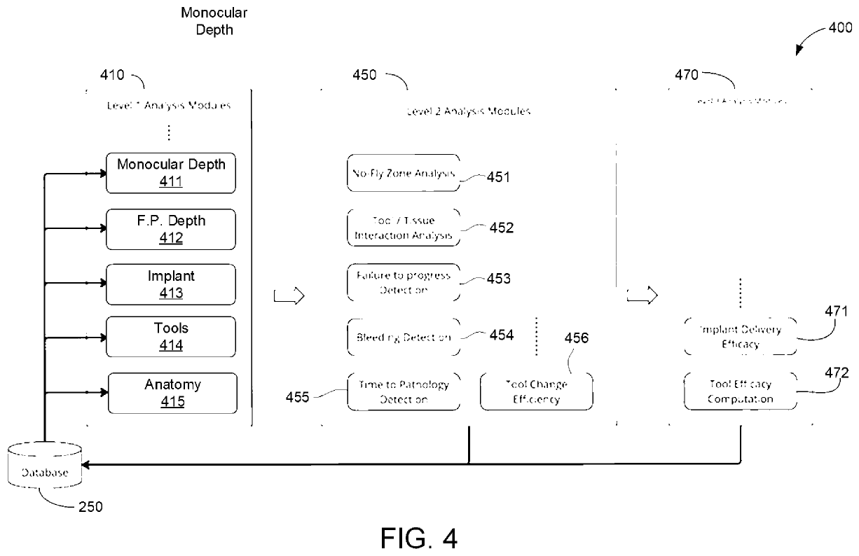

Next, in block 106, a secondary analysis of the surgical video (or video stream) is performed. The secondary analysis can use one or more outputs of the primary analysis as inputs. In some examples, the secondary analysis may include analysis by one or more processors executing one or more trained neural networks. The secondary analysis can provide further insights and critiques on the surgical videos. Secondary analysis is described in more detail below in conjunction with FIGS. 4-8.

FIG. 2 shows a block diagram of primary analysis modules 200 that may be associated with the primary analysis of a surgical video. In particular, the primary analysis modules 200 can include different modules that can analyze a live or recorded surgical video, such as a surgical video 202 (in some examples, the surgical video202 may be referred to as a video stream). The surgical video 202 may also include metadata 204. The metadata 204 may include any data that may be associated with the surgical video 202. Examples of metadata 204 can include timestamps, metadata regarding the surgery, patient demographics and the like. In some examples, the timestamps may be used by other modules to identify events, procedures, or surgical tools that are noted or recognized within the surgical video 202. A timestamp may be a unique identifier that may be associated with a particular frame of the surgical video 202.

The surgical video 202 may be processed by an artificial intelligence pipeline 206. The artificial intelligence (AI) pipeline 206 may include one or more neural networks that may be executed by one or more processors to perform one or more of the operations described herein. In some cases, execution of a neural network may include execution of a neural network that has been trained to perform a task or recognize an image, as described herein.

The AI pipeline 206 can include an input video preprocessing module 210 to preprocess the surgical video 202. In some examples, the input video preprocessing module 210 can center crop the surgical video 202. For example, the input video preprocessing module 210 can remove black space surrounding one or more objects centrally located within a field of view (FOV) of the surgical video 202. The FOV of a video may refer to any areas that may be within a central portion of the surgical video 202. In some examples, removing black space may include removing black space around the central portion of the view with removing any non-important image content.

In some examples, the input video preprocessing module 210 can resize the surgical video 202. For example, the input video preprocessing module 210 can scale the surgical video 202 to a size of approximately 256×256 pixels. In other implementations, other sizes are possible. In some variations, the surgical video 202 may be resized to a different shape of 320×320 pixels, 448×448 pixels, or other sizes, based on the step in the processing.

In some examples, the input video preprocessing module 210 can remove color information from the surgical video 202. For example, some subsequent processing steps or modules may include a neural network trained using black and white images. Thus, color information within the surgical video 202 may impede subsequent processing. The input video preprocessing module 210 may change or modify color information in the surgical video 202. In some examples, the input video preprocessing module 210 can convert colored image information of the surgical video 202 (RGB, YUV, or other color spaces) to a grayscale color space. In some examples, the input video preprocessing module 210 can remove color information after resizing. In some other examples, the input video preprocessing module 210 can remove color information prior to any resizing operations.

AI pipeline 206 may also include an inside/outside detection module 212 to determine whether contents of the surgical video 202 are inside or outside a body. Such a determination may be useful for other modules, particularly those based on recognizing operations or tools. Recognizing a tool or anatomy outside of the body may not be useful. In some examples, a processor or processing node may execute an artificial intelligence program or neural network to determine or detect when the surgical video 202 shows images from inside a body.

The AI pipeline 206 may include primary AI modules 240 that can provide some or all of the preliminary analysis of the surgical video 202. In some examples, the primary AI modules 240 can include an FOV quality module 241, a monocular depth estimation module 242, a surgical procedure recognition module 243, a surgical action recognition module 244, a view recognition module 245, an implant recognition module 246, a tool recognition module 247, a pathology recognition module 248, and an anatomy recognition module 249. Outputs of any of the primary AI modules 240 can be referred to as intermediate assessment data. The intermediate assessment data may be used by other modules to perform further, deeper analysis on the surgical video 202. In general, the outputs from any of the primary AI modules 240 may determine any notable clinical aspects. The clinical aspects may be identified with text labels, masks (video masks), and or timestamps associated with particular video frames of the surgical video 202.

The FOV quality module 241 can provide a quality assessment of the surgical video 202. In some examples, the FOV quality module 241 may output one or more text labels and associated video timestamps (video frame identifiers) associated with image quality. For example, the FOV quality module 241 may output blurry, bloody, and/or excessive debris labels along with the video timestamps of the video associated with the labels. Image quality may affect the ability of other modules to process or review the surgical video 202. In some embodiments, the FOV quality module 241 may include one or more neural networks trained to identify various image quality characteristics, including blurry images, blood within an image, and excessive debris within an image.

The monocular depth estimation module 242 may provide or generate a heatmap of an anatomical structure. The heatmap can indicate a perceived depth or distance from the camera. In some examples, more distant regions are depicted with darker tones and more proximal regions are depicted with brighter tones. Thus, the output of the monocular depth estimation module 242 may be tonal image. In some embodiments, the monocular depth estimation module 242 may include one or more neural networks trained to determine perceived depth.

FIG. 3A shows an image of a bony structure 300 and a corresponding anatomical structure heatmap 320. The bony structure 300 shows darker regions indicating areas that are farther away from the viewer. The cavity in the bony structure 300 is another example with darker regions being more distal from the viewer. The heatmap 320 show corresponding shaded areas that may be farther away from the view.

FIG. 3B shows another image of a bony structure 350 that includes a surgical tool 355. The monocular depth estimation module 242 may generate a corresponding anatomical structure heatmap 370 that includes the surgical tool 355.

The surgical procedure recognition module 243 may be used to determine whether a surgical procedure has been recognized within the surgical video 202. In some examples, the surgical procedure recognition module 243 may output a text label the indicates which particular surgical procedure has been recognized as well a timestamp indicating when the surgical procedure appears in the surgical video 202. In some examples, the surgical procedure recognition module 243 may include one or more neural networks that have been trained to recognize any feasible surgical procedure and determine any associated video timestamps.

The surgical action recognition module 244 may be used to determine whether any surgical procedure has been performed. Thus, while the surgical procedure recognition module 243 can recognize that a surgical procedure has been identified, the surgical action recognition module 244 can determine whether the surgical procedure has been performed and/or completed. The surgical action recognition module 244 may output text labels and video timestamps that indicate whether any surgical action has been recognized as well as the video frame that includes the recognized surgical action. In some examples, the surgical action recognition module 244 may include one or more neural networks that have been trained to recognize the performance and/or completion of any feasible surgical procedure as well as determine the associated video timestamps.

The view recognition module 245 may be used to indicate a recognized view in the surgical video 202. In some examples, a surgery can be considered to be a sequence of actions where the surgeon treats a pathology through the sequence of actions. These actions or activities may be bookended by distinct scenes, especially at the end of a procedure. These distinct scenes form visual landmarks to aid in the evaluation and/or understanding of a surgical procedure. For example, when a surgeon completes a ‘site preparation,’ surgical best practices indicate that the region ‘looks’ a given way. The view recognition module 245 can recognize various scenes and output an associated text label. The view recognition module 245 can also output video timestamps indicating where in the surgical video the recognized view appears. The view recognition module 245 may include one or more neural networks trained to recognize various scenes and also output corresponding text labels and timestamps.

The implant recognition module 246 can indicate whether the surgical video 202 includes any feasible recognized implant. Implants can include any hard or soft implant, replacement bone structure or the like. The implant recognition module 246 can output a text label that includes the name of the recognized implant. The implant recognition module 246 may include one or more neural networks trained to recognize various implants and also output the corresponding text labels and associated timestamps.

The tool recognition module 247 can indicate whether the surgical video 202 includes any feasible surgical tool. Surgical tools, scalpels, retractors, probes, and the like may be used in a variety of surgical procedures. In some examples, the tool recognition module 247 may include one or more neural networks trained to recognize any feasible surgical tool and also output a corresponding text label and a mask that outlines the recognized surgical tool. The tool recognition module 247 may also output any timestamps associated with the recognition of the surgical tool.

The pathology recognition module 248 can indicate whether the surgical video 202 includes any recognized pathology. A pathology can include any feasible disease or other physical malady. In some examples, the pathology recognition module 248 may include one or more neural networks that have been trained to recognize any feasible pathology and output a corresponding text label and mask that outlines or highlights the recognized pathology. The pathology recognition module 248 can also output a video timestamp associated with the recognized pathology.

The anatomy recognition module 249 can indicate whether the surgical video 202 includes any recognized anatomy. The recognized anatomy can include any feasible anatomy. In some examples, the anatomy recognition module 249 may include one or more neural networks that have been trained to recognize any feasible anatomy and output a corresponding text label and a mask that outlines or highlights the recognized anatomy. The anatomy recognition module 249 may also output a video timestamp associated with the recognized anatomy.

Outputs of some or all of the primary AI modules 240 may be stored in the datastore 250. The datastore 250 can be any feasible memory providing permanent or semi-permanent data storage.

In addition, outputs from some or all of the primary AI modules 240 may be used as inputs for further AI processing. Thus, some or all of the outputs of any of the primary AI modules 240 may be intermediate data for further processing. Further AI processing is described below in conjunction with FIGS. 4-8.

FIG. 4 shows a block diagram of a secondary analysis 400. The secondary analysis 400 may include processing blocks and/or modules that are associated with a further analysis (beyond the primary analysis modules 200 of FIG. 2) of the surgical video 202. Any of the outputs (intermediate assessment data) from the modules of FIG. 2 may be used as inputs for any of the modules of FIG. 4. As described above, outputs from the modules of FIG. 2 may be stored in the datastore 250. Thus, any data needed by any of the modules of FIG. 4 may be retrieved from the datastore 250.

The secondary analysis 400 may include level 1 analysis modules 410, level 2 analysis modules 450, and level 3 analysis modules 470. The level 1 analysis modules 410 may generate one or more interactive graphical representations of surgical information that has been detected within the surgical video 202. The graphical representations can distill complex surgical information into a compact format that advantageously enables quick and easy analysis of a surgeon's performance. In some examples, the graphical representations may be line graphs, however, other graphs are possible. In some implementations, the graphical representations may be interactively displayed on a monitor or other user interface. The user may expand (e.g., zoom into) any region of the graphical representation to examine any graphical data more closely. In some examples. The level 1 analysis modules 410 may include a monocular depth analysis module 411, a feature point depth analysis module 412, an implant analysis module 413, a tools analysis module 414, and an anatomy analysis module 415. In other examples, the level 1 analysis modules 410 may include more or fewer analysis modules.

In general, level 1 analysis modules 410 use outputs of the primary analysis modules 200 from the datastore 250. The outputs may include text labels, masks, and video timestamps. The level 1 analysis modules 410 can create a high-level view or summary of the surgery which can yield considerable insights. The outputs or results of the level 1 analysis modules 410 are typically depicted visually by line graphs (e.g., a graphical representation), though other visualizations are equally possible using any other feasible data analysis tools.

The generated graphs may include a graphical representation of the video's surgical information. The generated graphs may contain levels, or quantities, of presence of various surgically-related objects over time in any arbitrary video segment or the entire surgery video. Since the generated graphs are the output of inferences applied to each frame (from the application of any of the primary AI modules 240) and the subsequent information consolidation, in the form of computing the proportions of the field of view occupied by a given structure, the graphs are a very dense representation of the information content in the surgical video. The information in the output of the inferences is compressed by summing the area of each structure outputted by the AI module 240. This allows information regarding which structures are present, and the level of their presence, to be computed, but in a representation which is much smaller than the direct output than the AI module 240. This representation is smaller since there is simply one floating-point number which represents each structure rather than an entire image representing each structure. When collected across an entire video, one obtains a graph of the presences of each structure, and thus obtain a dense representation of the AI module outputs. Owing to its information density, any of the generated graphs can provide deep insights when analyzed by subject matter experts. This visualization also enables a clinically-relevant information to be understood at a glance due to the visual representation for the entire surgical video.

In some examples, to generate a graphical representation, any of the level 1 analysis modules 410 may use the output of the FOV quality module 241 on each frame in the surgical video 202. If a frame is determined to be a ‘bad’ frame (e.g., blurry, occluded, out-of-body, or any other type of poor image quality frame), any subsequent analysis module can simply exclude that given frame from further analysis and move to the next frame. On the other hand, if the frame is determined to be ‘good,’ then any of the processing described herein may proceed.

In some examples, segmentation masks or classification (text) labels from any of the primary AI modules 240 may be converted into percentages or detection Booleans for any surgical video frame. Percentages are calculated as a pixel area of a given object's mask divided by a total pixel area of the image. Regardless of the nature of any of the primary AI modules 240, the output of any of the level 1 analysis modules 410 may be an array of either decimal-valued levels or a binary presence for each class associated with the selected level 1 analysis module 410. For example, if the anatomy analysis module 415 is segmenting seven possible anatomy masks, there may be seven decimal quantities, i.e., a 7-dimensional vector, for every surgical video frame.

For example, the anatomy analysis module 415 may process the masks produced by the anatomy recognition module 249. The anatomy analysis module 415 can estimate relative proportions of the image (video frame) occupied by a recognized anatomical structure. The anatomy analysis module 415 can output a multi-dimensional vector with each dimension representing the relative proportion of the structure, corresponding to the dimension, in the image. These vector points may be plotted against their respective timestamps.

In some cases, smoothing may be applied to any of the generated graphical representations. Since the segmentation and classification models (e.g., any of the primary AI modules 240) often yield noisy outputs, especially over time for consecutive frames, a locally weighted polynomial regression algorithm (LOWESS) is applied. The LOWESS computes a polynomial weighted best fit line over a sliding window. LOWESS can drastically reduce visual noise and allows for model outputs to be visually interpretable. The output, a sequence of vectors with associated timestamps, is written to the datastore 250.

For example, any of the modules described herein can analyze any of the inferences of the primary AI modules 240 that have been written to the datastore 250. Each inference may generate a different graph or graphical representation. In some embodiments, each of the level 1 analysis modules 410 may work primarily with one of the primary AI modules 240.

The monocular depth analysis module 411 can determine the depth of anatomical structures and tools by using monocular depth estimation algorithms. In some examples, the monocular depth analysis module 411 may include one or more neural networks that have been trained to estimate the depth of objects appearing in the field of view. The depth heatmaps (from the monocular depth estimation module 242) are combined with anatomy and masks (from the tool recognition module 247). The depth of various recognized structures is plotted against time. The monocular depth analysis module 411 can output a vector corresponding to the relative depth of a given anatomical structure or tool from the surgical video 202. Since the anatomical structures are not normal to a field of view, a single estimate of depth might not be sufficient. Thus, each depth estimation could contain max, min, and mean depth estimates.

In some examples, the monocular depth analysis module 411 can estimate proper scoping positions. These estimated scoping positions could inform surgeons regarding the nature of tool tissue interactions. Certain tools, such as burr tools may need more caution when handled near cartilages. Any accidental contact of the sharp blades of the burr tool with cartilage could be harmful to the patient. Thus, the monocular depth analysis module 411 can determine distances between tools and parts of the patient.

The feature point (F.P.) depth analysis module 412 can use computer-vision-based feature points to infer feature depths. In some examples, the feature point depth analysis module 412 may include one or more neural networks that have been trained to infer or estimate the depth of objects appearing in the field of view. The feature point depth analysis module 412 first extracts feature points based on highly local corner detection. These feature points may include visual folds, boundaries, or textures within a current view that have local edges or corners. The feature point depth analysis module 412 maintains a buffer of these features for several video frames and uses these feature to detect whether the number of features in a given anatomical structure is dropping. When such a drop is detected at the same time as the increase in relative prominence of a given anatomical structure, the drop could indicate that the scope (field of view) is zoomed in and the apparent depth is small. The feature point depth analysis module 412 can operate on the principle that the spatial distribution of any feature points will be much more uniform for a “regular” frame as compared to a deleterious close-up frame, which will have few texture features on the close-up object.

As a best practice, surgeons should maintain the scope (camera) at proper distance from the target sites, to provide them an idea of the margins they should observe when treating pathologies. An experienced surgeon will be able advantageously to observe any graphical representations showing a feature point depth analysis and suggest corrective actions. Context for the surgeons' actions may be determined from other graphical representations (other level 1 analysis modules) and the output of the surgical action recognition module 244.

The implant analysis module 413 can generate a multi-dimensional vector for each input image (timestamped video frame), where each vector dimension corresponds to the presence or detection of a particular implant in the image. In some examples, the implant analysis module 413 may include one or more neural networks that have been trained to recognize implants appearing in the field of view. Unlike the anatomy masks (described below with respect to the anatomy analysis module 415), the vector for the implant analysis module 413 can consist of Boolean values which indicate the presence of a given implant. In some examples, the implant analysis module 413 can generate graphical representations that show efficient (timely) implant usage.

The tools analysis module 414 can generate a multi-dimensional vector for each frame in the surgical video 202, with each dimension corresponding to the presence of a particular surgical tool in the frame. In some examples, the tools analysis module 414 may include one or more neural networks that have been trained to recognize surgical tools appearing in the field of view. Unlike the anatomy masks, each dimension may be represented by a Boolean value that indicates the presence of a surgical tool.

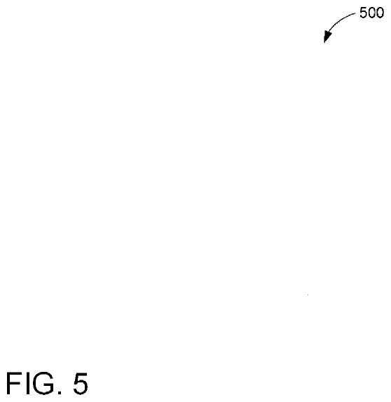

FIG. 5 shows an example graphical representation 500 based on the multi-dimensional vector described herein with respect to a shoulder procedure. The tools analysis module 414 can generate the graphical representation 500. In this example, the graphical representation 500 depicts very efficient surgery. Tool changes are minimal, the RF tool is used a lot at the start of the procedure, indicating that the surgeon prefers to debride, probe, and test various structures using just a single tool. The saw-tooth pattern in the latter half of the image shows efficient anchor insertion and suture passing.

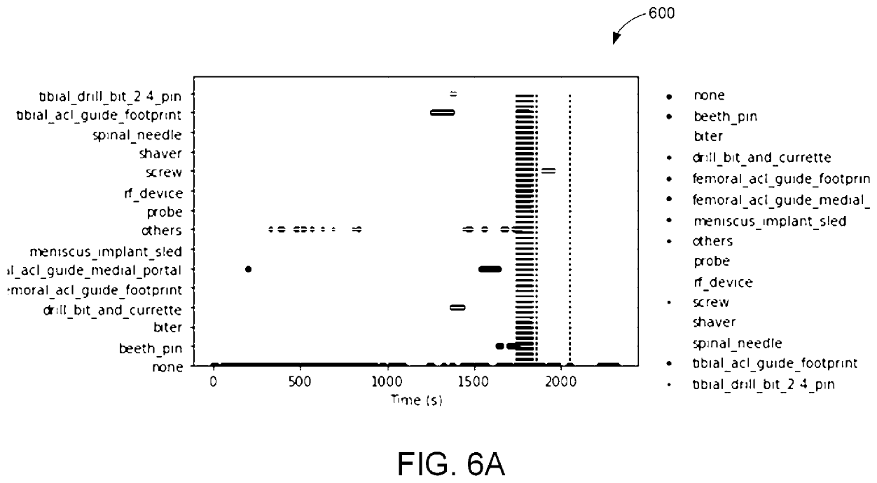

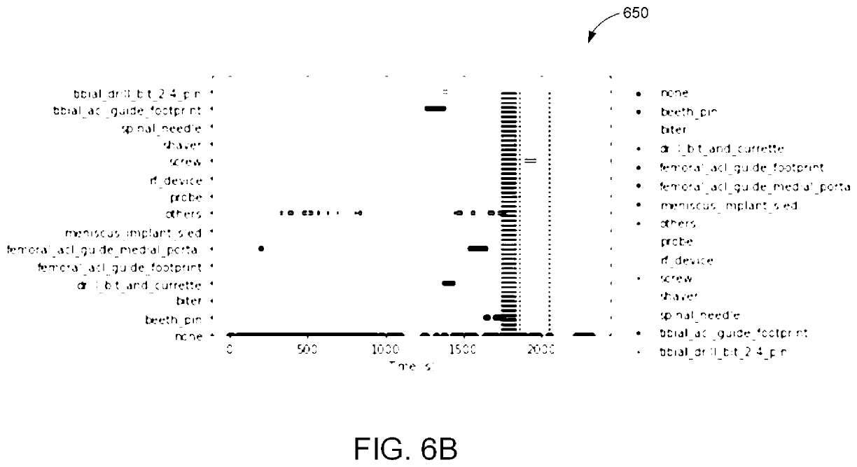

FIG. 6A shows another example graphical representation 600 showing surgical tool use with respect to time. As before, the tools analysis module 414 can generate the graphical representation 600. In this example, the horizontal axis represents time. Each horizontal level, on the vertical axis, represents the presence or absence of any particular tool. The graphical representation 600 combines the outputs of tool recognition module 247 and implant recognition module 246 (in this case suture recognition). In some examples, the red rectangle, appearing approximately at time index 1750, can indicate the presence of a suture in the frame regardless of which tool is detected. As is noted, the surgery ends with the surgeon presumably cleaning loose tendons with a shaver.

In some examples, the tools analysis module 414 can also output information regarding image quality (from the FOV quality module 241) in parallel with any other output. FIG. 6B is an example graphical representation 650 showing area with poor FOV quality. For example, when the FOV quality module 241 indicates the presence of poor-quality video frames, then the surgical video 202 may indicate poor technique (inadequate use of fluid pumps to clear the field of view). The gray rectangular overly in the graphical representation 650 indicates instances in time where the field of view was sub-optimal.

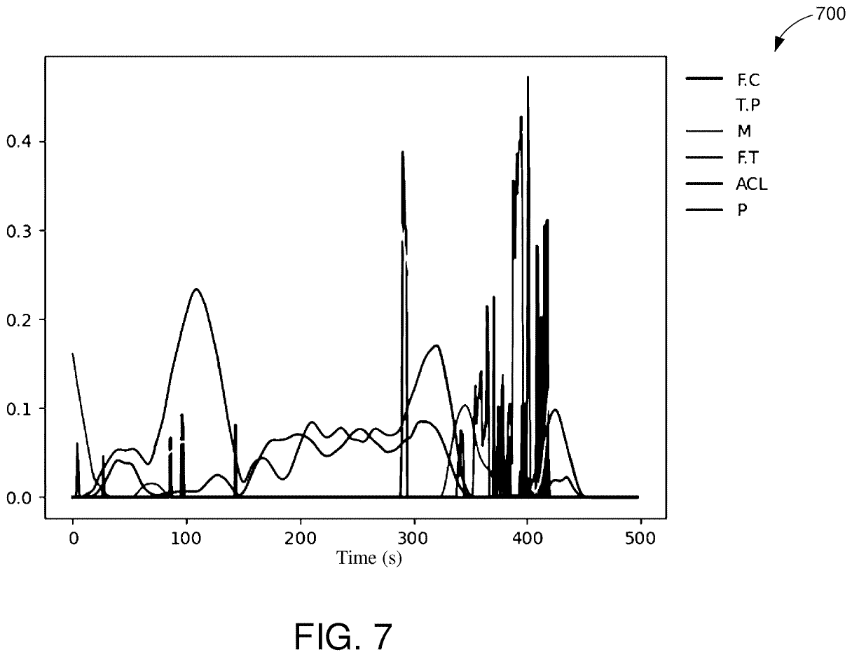

Returning to FIG. 4, the anatomy analysis module 415 can output a multi-dimensional vector for each input frame, with each dimension corresponding to the relative coverage of the anatomical structure in the image. In some examples, the anatomy analysis module 415 may include one or more neural networks that have been trained to recognize various anatomical structures appearing in the field of view. FIG. 7 is an example of a graphical representation 700 for a surgical procedure in the knee (meniscal repair) based on the multi-dimension vector from the anatomy analysis module 415. The graphical representation 700 may be generated by the anatomy analysis module 415. In this example, the graphical representation 700 is a plot of the multi-dimensional vector over the course of a relatively short video segment. Note, the abbreviations F.C, T.P., M, R.T, ACL, and P can correspond to Femoral Condyle, Tibial Plateau, Meniscus, Femoral Trochlea, Anterior Cruciate ligament, and patella, respectively.

In some examples, the graphical representation 700 can show time required by the surgeon to arrive at the target site (which in this example is the meniscus) can be a significant factor for assessment.

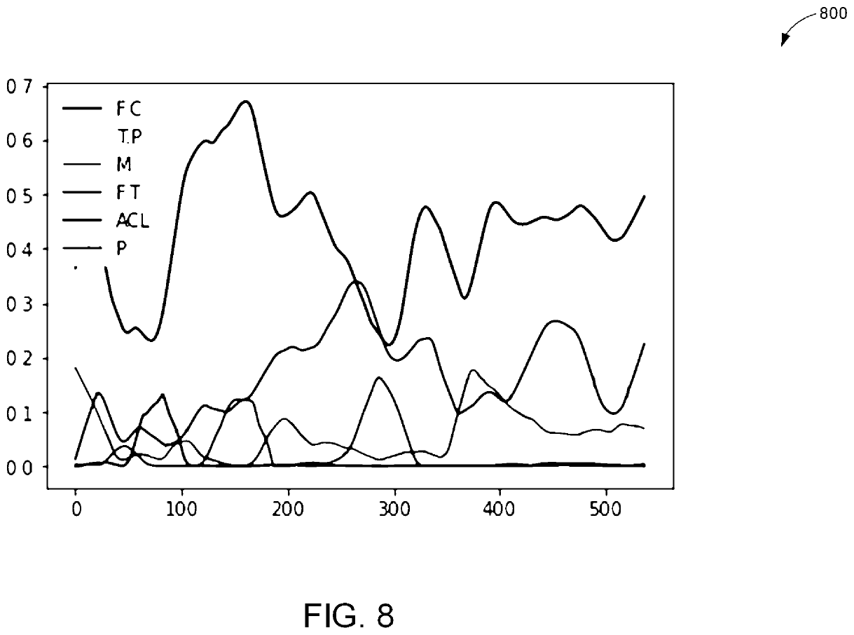

FIG. 8 is an example graphical representation 800 of a surgical procedure for anterior cruciate ligament reconstruction. In this example, the relative preponderance of the femoral condyle indicates that the surgeon's primary area of interest is the femoral condyle.

Returning to FIG. 4, the level 2 analysis modules 450 may include a no-fly zone analysis module 451, a tool/tissue interaction analysis module 452, a failure to progress detection analysis module 453, a bleeding detection analysis module 454, a time to pathology detection analysis module 455, and a tool change efficiency module 456. In other examples, the level 2 analysis modules 450 may include more or fewer analysis modules.

In some examples, the level 2 analysis modules 450 may detect specific events. Some level 2 analysis modules 450 may be developed with insights from subject matter experts. That is, some level 2 analysis modules 450 can use a combination of rules, deep and machine learning techniques to perform analysis. The following modules suggest how further extensions and/or insights can be derived from any feasible data in the datastore 250 of FIG. 2. All of the level 2 analysis modules can write their outputs along with appropriate metadata, video timestamps, etc., into the datastore 250.

The no-fly zone analysis module 451 can analyze a surgical video and determine if a specific surgical tool was operated in a region for which it is not intended. The no-fly zone analysis module 451 can use outputs from the monocular depth analysis module 411, the feature point depth analysis module 412, the FOV quality module 241 (to detect for the presence of debris), the tools analysis module 414, and anatomy analysis module 415. The no-fly zone analysis module 451 can use the presence of loose debris, obtained from the FOV quality module 241 to compensate for instances where the surgeon intends to operate the burr in a given region. For instance, the deliberate and persistent operation of a burr tool can be discerned by the presence of debris in the field of view, it should be assumed that this is a deliberate action on behalf of the surgeon and an alert should not be provided.

The tool/tissue interaction analysis module 452 can detect unusual tool-tissue interactions. In some examples, the tool/tissue interaction analysis module 452 functionality can overlap no-fly zone analysis module 451 functionality. In general, tool/tissue interaction analysis module 452 can use rules and signal processing techniques to detect unusual tool-tissue interactions. Surgery/procedure specific rules are constructed to indicate preferred tools for various stages of a given surgical procedure. These rules are continually asserted by tool/tissue interaction analysis module 452. Rules need not be constructed for every conceivable situation; it is sufficient to simply flag unusual cases for further analysis by a subject matter expert or a power user. As used herein, rules (e.g., “business rules”) may be asserted by a rules execution engine on demand. The context, i.e., the data supplied to the rules, could change with each instance.

In some examples, if a surgeon returns to an area repeatedly with the same tool, it might indicate that the surgeon is struggling to prepare the repair site properly. By the same token, if the surgeon returns to the same area with an unusual tool for the context, it might indicate that the surgeon is recovering from an error or a mishandled implant. In these and other cases, the tool/tissue interaction analysis module 452 can flag these events.

The failure to progress detection analysis module 453 can detected a stalled or delayed surgical procedure. In some examples, when any of the level 1 analysis modules 410 do not show any meaningful change over time, then the failure to progress detection analysis module 453 may determine that the surgery has been stalled. The failure to progress detection analysis module 453 module can apply basic signal processing algorithms to any of the outputs of any of the level 1 analysis modules 410.

Notably, some surgeons may pause to prepare grafts for implantation. In some other cases, the staff might be trying to locate a specific implant due to a newly discovered pathology. In any case, the alerts generated by the failure to progress detection analysis module 453 will be used to trigger some inspection and analysis with regard to these pauses.

The bleeding detection analysis module 454 can detect a change in the color hue of a surgical video towards red beyond the threshold, i.e., related to the inflammation on anatomical structures. In this manner, the bleeding detection analysis module 454 can output a label to indicate the presence of bleeding in the frame,

In some examples, to avoid conflicting signals from the introduction of the tools and changes in any hue histogram, a secondary analysis module can consider the changes in hue histograms to the timestamps when tools are introduced into the FOV, and compensates for the corresponding changes. The bleeding detection analysis module 454 can also consider the output of the anatomical and pathology masks, evaluate the hue histograms of these masks to detect the presence of inflamed tissues which could tip the color balance towards red.

In some examples, the time to pathology detection analysis module 455 can determine a time between the detection of a pathology to the detection of a specific tool to treat the pathology. In some cases, surgery metadata may be examined as well as the timestamp associated with the appearance or detection of a target pathology for the surgery type indicated by a procedure code. The time when the target pathology is treated may also be determined. Treatment is considered to have started when a specific tool is detected in the field of view over the target pathology and a predetermined surgical action corresponding to the activity is detected.

While a direct observation of the “Time to Pathology” might not be very meaningful, relative performance between surgeons, controlled for a given surgery type could be useful. Similarly, it may also be possible to track the gains in surgical performance over time.

The tool change efficiency module 456 can use one or more machine learning algorithms (trained neural networks) to discover unusual delays in the time it takes to switch between certain tools. The tool change efficiency module 456 can use outputs from the tool recognition module 247. Surgery metadata can be used to determine surgical complexity. The tool change efficiency module 456 module can output the labels of tools which take longer than an average time to position and use.

The level 3 analysis module 470 may include an implant delivery efficacy module 471 and a tool efficacy computation module 472. In other examples, the level 3 analysis modules 470 may include more or fewer analysis modules.

In general, the level 3 analysis modules 470 can provide higher level insights by aggregating and analyzing Level 1 and Level 2 analyses from multiple surgeries, surgeons, tools, implants, etc. The level 3 analysis modules 470 may be performed entirely through traditional data analysis tools.

When considered in the context of a given surgeon, insights from the level 3 analysis modules 470 can assess the performance of a surgeon, identify weaknesses/areas for improvement as evidenced by the outputs of the level 1 analysis modules 410 and level 2 analysis modules 450. In some cases, considered in the context of multiple surgeons within a given surgery type, these insights could drive financial decisions.

In general, the level ‘3’ modules are activated to analyze patterns in surgical activities after controlling for similar pathologies and surgeons' proficiencies. The system aggregates and analyzes the output of level ‘2’ modules across patients who present similar pathologies for specific tasks, such as the delivery of implants, module 471. If level ‘2’ modules indicate slowdowns, across multiple surgeons regardless of skill/seniority level, it could be concluded that a given implant is inherently more complicated to install. This insight could be used to trigger additional training or a quest for alternatives. While a simple time study analysis is possible, unless it is controlled for similar pathologies and surgeon proficiency levels, it is not actionable. Similarly, module 472 could be used to look for time taken to engage and complete the tasks with a given tool. Again, this analysis should control for pathologies and surgeon skill levels before the insights can be considered to be actionable.

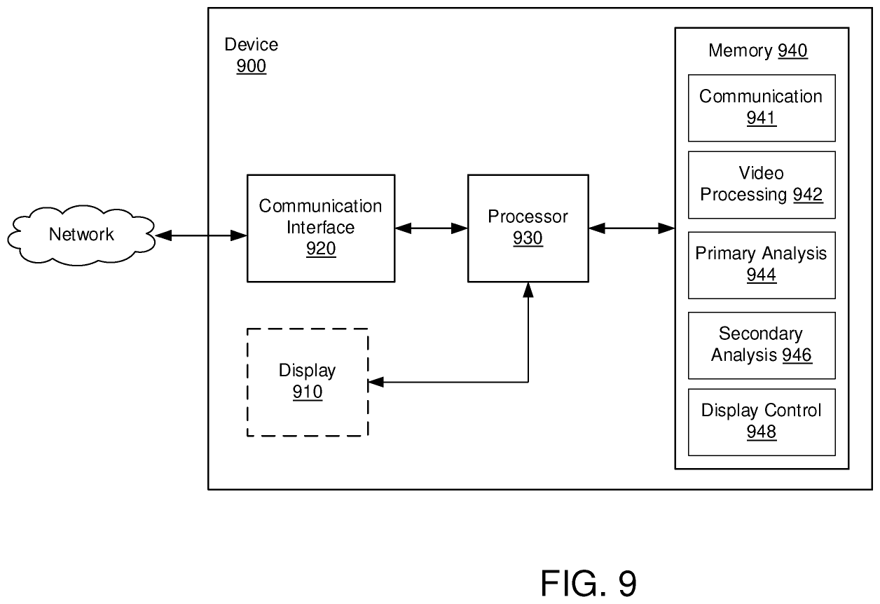

FIG. 9 shows a block diagram of a device 900 that may perform some or all of the operations described herein. The device 900 may include a display 910, a communication interface 920, a processor 930, and a memory 940.

The communication interface 920, which may be coupled to a network (such as the internet or any other feasible network) and to the processor 930, may transmit signals to and receive signals from other wired or wireless devices, including remote (e.g., cloud-based) storage devices, cameras, processors, compute nodes, processing nodes, computers, mobile devices (e.g., cellular phones, tablet computers and the like). For example, the communication interface 920 may include wired (e.g., serial, ethernet, or the like) and/or wireless (Bluetooth, Wi-Fi, cellular, or the like) transceivers that may communicate with any other feasible device through any feasible network. In some examples, the communication interface 920 may receive surgical video or live stream surgical video data.

The display 910, which is coupled to the processor 930, may be used to display any of the results from any of the operations described herein. In some examples, the display 910 may be optional with respect to the device 900, as shown here with dashed lines. For example, the functionality of the display 910 may be provided by a separate display coupled through a network. The separate display can be included within a separate computing device (laptop, tablet computer, cellphone, or the like).

The display 910 can display any of the graphical representations output from any of the analysis modules, including any of the level 1 analysis modules 410 of FIG. 4. In some implementations, the display 910 may also include a touch-sensitive user interface that can receive a user's touch input to move or manipulate the information rendered on the display 910. For example, a user can zoom or expand some or all of any graphical representation. In some aspects, the user can control some or all of the operations of the device 900 through the display 910, including initiating any analysis on any surgical video data.

The processor 930, which is coupled to the memory 940, may be any one or more suitable processors capable of executing scripts or instructions of one or more software programs stored in the device 900 (such as within memory 940).

The memory 940 may also include a non-transitory computer-readable storage medium (e.g., one or more nonvolatile memory elements, such as EPROM, EEPROM, Flash memory, a hard drive, etc.) that may store the following software modules:
a communication module 941 to transmit and receive data through the communication interface 920;
a video processing module 942 to process surgical video data;
a primary analysis module 944 to perform a primary analysis with respect to any surgical video data;
a secondary analysis module 946 to process a secondary analysis with respect to any surgical video data; and
a display control module 948 to control operations associated with the display 910.
   Each module includes program instructions that, when executed by the processor 930, may cause the device 900 to perform the corresponding function(s). Thus, the non-transitory computer-readable storage medium of memory 940 may include instructions for performing all or a portion of the operations described herein.

The processor 930 may execute the communication module 941 to communicate with any other feasible devices. For example, execution of the communication module 941 may enable the device 900 to communicate via cellular networks conforming to any of the LTE standards promulgated by the 3^{rd} Generation Partnership Project (3GPP) working group, Wi-Fi networks conforming to any of the IEEE 802.11 standards, Bluetooth protocols put forth by the Bluetooth Special Interest Group (SIG), Ethernet protocols, or the like. In general, execution of the communication module 941 may enable the device 900 to communicate with any wired or wireless networks. In some other embodiments, execution of the communication module 941 may implement encryption and/or decryption procedures. Further, the device 900 may transmit any data, including graphical representations, to other devices or machines for review by other users.

The processor 930 may execute the video processing module 942 to receive, obtain, and/or process surgical video. For example, execution of the video processing module 942 can cause the processor 930 to perform any of the video processing as described with respect to the input video preprocessing module 210 of FIG. 2. In some examples, execution of the video processing module 942 can scale, crop, and/or remove color from any surgical video.

The processor 930 may execute the primary analysis module 944 to perform any analysis including operations described with respect to the primary AI modules 240 of FIG. 2. Thus, execution of the primary analysis module 944 may cause the processor to perform any operations associated with the FOV quality module 241, the monocular depth estimation module 242, the surgical procedure recognition module 243, the surgical action recognition module 244, the view recognition module 245, the implant recognition module 246, the tool recognition module 247, the pathology recognition module 248, and the anatomy recognition module 249. In some examples, the primary analysis module 944 may include one or more trained neural networks to perform any operations for any of the above-mentioned modules.

The processor 930 may execute the secondary analysis module 946 to perform any analysis including operations described with respect to the secondary analysis 400 of FIG. 4. Thus, execution of the secondary analysis module 946 may cause the processor 930 to perform any operations associated with the monocular depth analysis module 411, the feature point depth analysis module 412, the implant analysis module 413, the tools analysis module 414, and the anatomy analysis module 415. In addition, execution of the secondary analysis module 946 may also cause the processor 930 to perform any operations associated with the no-fly zone analysis module 451, the tool/tissue interaction analysis module 452, the failure to progress detection analysis module 453, the bleeding detection analysis module 454, the time to pathology detection analysis module 455, and the tool change efficiency module 456.

The processor 930 may execute the display control module 948 to control operations associated with the display 910 or any other display connected through the network (not shown). For example, execution of the display control module 948 may cause the processor 930 to render graphs (including any graphical representations described herein) on the display 910. Execution of the display control module 948 may cause the processor 930 to shrink (zoom out), enlarge (zoom in), or pan around (move in relative x and y axis directions) any graphical representation. In some examples, the display 910 may include a touch interface to receive user input. Thus, execution of the display control module 948 may cause the processor 930 to change data on the display 910 based on any received touch input.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of providing a surgical assessment, the method comprising:
receiving a video of a surgical procedure, wherein the video includes a plurality of frames, each frame having a unique timestamp;
generating intermediate assessment data from the video of the surgical procedure based on an execution of one or more trained neural networks configured to recognize one or more clinical aspects within the video, wherein the intermediate assessment data includes one or more text labels identifying the clinical aspect; and
interactively displaying a graphical representation of surgical information included in the video, based on the intermediate assessment data, further comprising a tool change efficiency analysis based on labels of tools within the intermediate assessment data.

2-25. (canceled)
